(19) United States
(12) Patent Application Publication
(10) Pub. No.: US 2006/0293548 A1
(43) Pub. Date: Dec. 28, 2006

(54) Metathesis catalyst and process

(76) Inventors: Alta Spamer, Vanderbijpark (DE); Jan Mattheus Botha, Sasolburg (ZA); Chart Van Schalkwyk, Sasolburg (ZA); Denzil James Moodley, Sasolburg (ZA); Thamsanga Ian Dube, Sasolburg (ZA)

Correspondence Address: HAHN AND MOODLEY, LLP, P.O. BOX 52050, MINNEAPOLIS, MN, 55402, US

(21) Appl. No.: 10/524,795

(22) PCT Filed: Aug. 14, 2003
(86) PCT No.: PCT/ZA03/00111

§ 371 Date: May 26, 2005

(30) Foreign Application Priority Data
Aug. 16, 2002 (ZA) 2002/6572

(51) Int. Cl. C07C 6/00; B01J 21/00

(52) U.S. Cl. 585/643; 502/254

(57) ABSTRACT

The invention provides a method of preparing a metathesis catalyst, the method including the steps of mixing a transition metal oxide containing aqueous solution having a pH of 9 or higher with a carrier. The water is then removed from the mixture by means of evaporation to provide a metathesis catalyst.

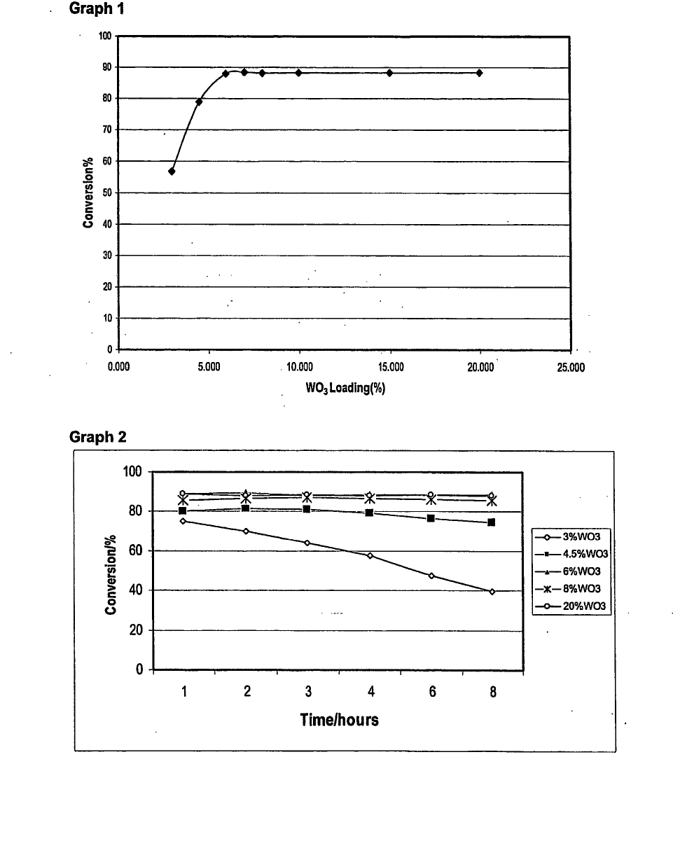

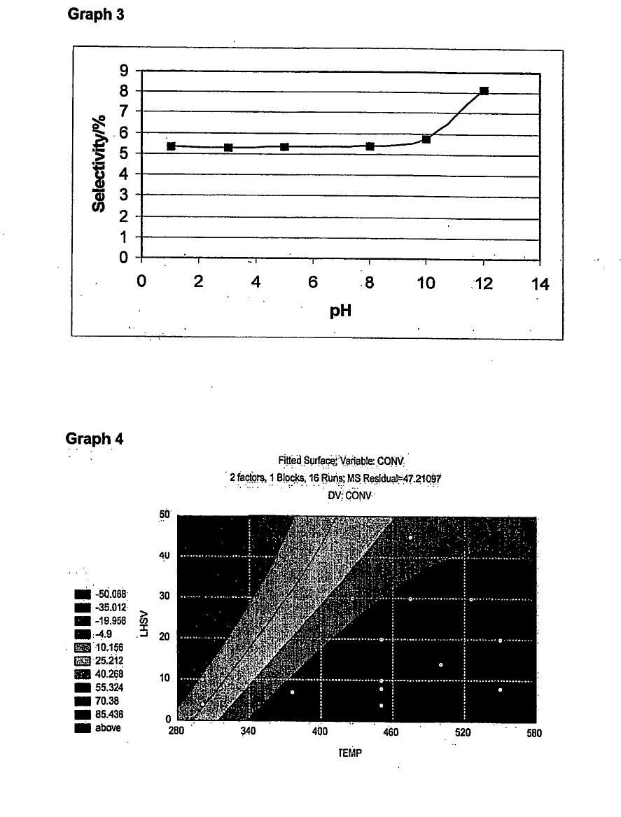

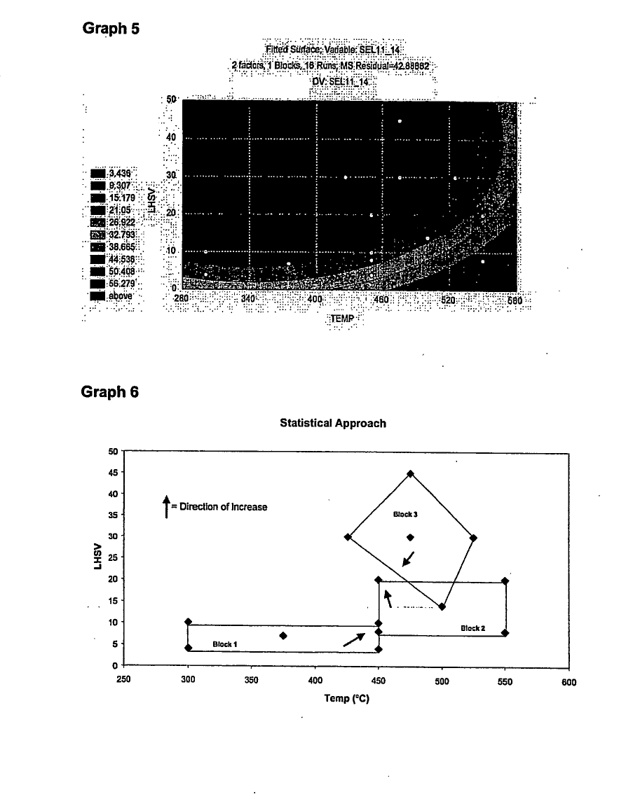

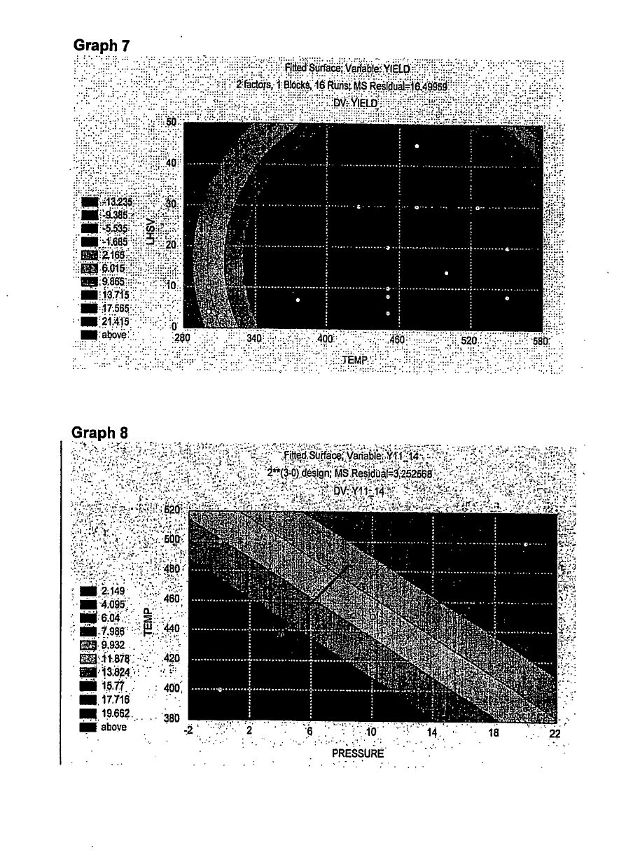

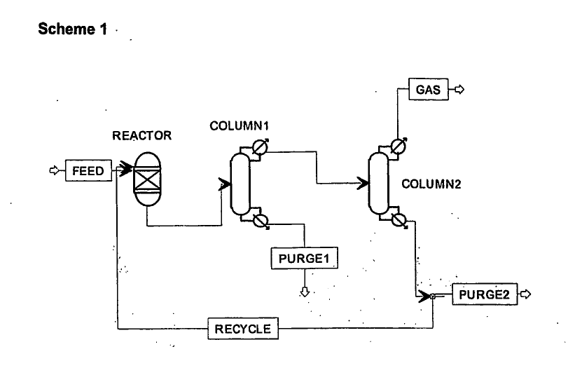

### TECHNICAL FIELD OF THE INVENTION

This invention relates to a metathesis catalyst, a method of preparing a metathesis catalyst, a metathesis process and a product produced by the metathesis process.

### BACKGROUND TO THE INVENTION

Metathesis, also known as olefin disproportionation, is a well-known process for facilitating carbon transfer between or among one or more olefins of an olefinic feed stream. Metathesis is a commercially valuable method for converting lower value olefinic streams into higher value olefinic streams. For example, the first and well-known metathesis process, the Triolefin process of Phillips Petroleum Co., was developed for transforming a stream of short chained olefins comprising propylene into a higher value stream of ethylene and 2-buthene using WO₃ on a silica (SiO₂) carrier as a catalyst. It is known that, with the use of catalysts other than WO₃/SiO₂, a 1-pentene stream can be transformed into a stream of 4-octene, with high selectivity, and ethylene using a molybdenum nitrosyl or carbonyl complex as a catalyst. The metathesis of longer chained C₆ olefins and even higher has been disclosed in U.S. Pat. No. 5,162,597 using WO₃ on an AL₂O₃ carrier as a catalyst.

The known method of preparing WO₃/SiO₂ catalyst comprises wet impregnation by adsorbing negatively charged oxyanion polytungstate onto silica gel. The silica gel can be polarised or positively charged by lowering the pH to below its iso-electric point of between about 1 and 2. It has been shown that a variety of tungsten oxyanion species containing one, six and twelve tungsten atoms can be present in aqueous solution, which can be controlled, to some extent, by the pH of the solution. At a pH of below about 6, a six and twelve tungsten atom species are dominant and below a pH of about 4 a twelve tungsten species is dominant. The applicants have found that the active tungsten sites of the WO₃/SiO₂ catalysts prepared at a low pH are randomly distributed on the surface of the SiO₂ and that clusters are formed at high loading of WO₃ loading of more than about 6 wt % on SiO₂. These clusters are inactive for metathesis. The applicants further found that, disadvantageously, that catalysts prepared at pH below the iso electric point have a low conversion and selectivity towards linear olefin or primary metathesis products of the metathesis of longer chained olefinic feed streams. The applicants also found that WO₃ loading of more than about 6 wt % on SiO₂, provides no significant increase in conversion rate although it leads to a lower selectivity, due to increased Brφnsted acidity, towards linear olefin products or primary metathesis products of the metathesis of longer chained olefinic feed: streams. The formation of secondary metathesis products is a result of isomerisation of the olefinic feed stream followed by metathesis. It is therefore important to lower the degree of Brφnsted acidity in order to limit the isomerisation reactions.

For linear alpha-olefinic feed streams, primary metathesis product shall be understood to be linear olefins having 2n−2 carbons with the double bond at the n−1 position, with n being the carbon number of the predominant linear alpha olefin of the olefinic feed stream.

A further disadvantage of these catalysts are the relatively high operating temperatures of up to 600° C., which lead to side reactions such as cracking, oligomerization, aromatisation, dehydrogenation etc.

However, these catalysts have certain inherent advantages over other commercially available metathesis catalysts like MoO₃/Al₂O₃ and Re₂O₇/Al₂O₃ that makes it attractive for commercial applications. Firstly, it has a considerable resistance to poisons as might be expected from the high operating temperatures, typically between 300-600° C., secondly it has a long on-line lifetime compared to Mo- and Re-based metathesis systems, due to its resistance to poisons, and thirdly it can be regenerated without negative effect on catalyst structure.

It is therefore an object of this invention to provide a WO₃/SiO₂ metathesis catalyst having all of its inherent advantages together with a relatively high conversion rate and selectivity, and an improved and optimised metathesis process using such a catalyst.

An example of an attractive application of such a WO₃/SiO₂ metathesis catalyst is a conversion process of alpha-olefins (C₅ to C₁₀), into longer chain, higher value olefins.

### GENERAL DESCRIPTION OF THE INVENTION

According to a first aspect of the invention there is provided a catalyst for metathesis of an olefinic feed stream, which includes:

a transition metal oxide; and

a carrier, the transition metal oxide being deposited onto the carrier from an aqueous solution of tungstate anions at a pH of more than about 9.

The transition metal oxide may be tungsten oxide and the carrier may be silica.

It will be appreciated that the deposits form the catalytically active sites on the carrier.

The tungsten oxide may be deposited onto the carrier from an aqueous solution of tungstate anions at a pH of more than about 10.

The tungsten oxide may be deposited onto the carrier from an aqueous solution of tungstate anions at a pH of about 12.

The catalyst may be a heterogeneous catalyst.

The catalyst may further be characterised in that the tungsten oxide deposits are substantially uniformly distributed on the surface of the carrier.

The catalyst may even further be characterised in that most of the tungsten oxide deposits are substantially amorphous.

The catalyst may also be characterised in that at least a portion of some of the tungsten oxide deposits are in the form crystallites of less than about 135 Å across on the surface of the carrier.

The tungsten oxide may be from about 4 to 10 wt % on SiO₂.

The tungsten oxide may be from about 5 to 8 wt % on SiO₂.

According to a second aspect of the invention, there is provided a method of preparing a metathesis catalyst, the method including the steps of:

mixing a transition metal oxide containing aqueous solution having a pH of about 9 or higher with a carrier; and

removing water from the mixture by means of evaporation.

The transition metal may be silica and the transition metal may be tungsten.

The aqueous solution may contain tungsten in the form of ammonium metatungstatehydrate and/or ammonium tungstate.

The concentration of the ammonium metatungstatehydrate and the mass of the silica may be selected such that the WO₃ on the SiO₂ is from about 4 to 10 wt %.

The concentration of the ammonium metatungstatehydrate and the mass of the silica may be selected such that the WO₃ on the SiO₂ is from about 5 to 8 wt %.

The aqueous solution of ammonium metatungstatehydrate may have a pH of more than about 10.

The aqueous solution of ammonium metatungstatehydrate may have a pH of about 12.

Excess water may be removed by evaporation at about 80° C. under reduced pressure. It will be appreciated that the temperature and pressure may be substantially varied to evaporate the excess water.

Further water may be removed after removal of the excess water by drying the residue at about 110° C. for about 12 hours, then by raising the temperature at a rate of about 1° C. every minute up to about 250° C., maintained at about 250° C. for about two hours and then by raising the temperature at a rate of about 3° C. every minute up to about 550° C.

The residue may then be calcined at about 550° C. for about 8 hours.

It will also be appreciated that for the removal of further water and the calcination the temperature and time may be substantially varied and substantially the same result obtained.

The calcination step substantially removes NH₃, ensures that the oxidation state of the tungsten is mostly 6+ and ensures that the tungsten oxide is bound to the carrier.

The pH of the aqueous solution may be adjusted before or during the mixing step by adding an acid such as nitric acid or by adding an alkali such as ammonium hydroxide.

According to a third aspect of the invention, there is provided a metathesis process, which includes the step of:

contacting a C₅ and/or higher olefinic feed stream with a catalyst for metathesis as described above at a temperature of between about 350° C. and 600° C.

The process may include a step of activating the catalyst at about 500 to 700° C. for about 8 hours in an inert atmosphere.

The olefinic feed stream may be selected such that the process yields C₁₀ to C₁₈ olefins. The C₁₀ to C₁₈ olefins are also known as the detergent range olefins and may be used to manufacture detergents, diesels, drilling fluids, synthetic lubricants and other down stream products. The feed stream may include C₅ to C₁₀ alpha olefins.

The feed stream may be contacted with the catalyst at a LHSV (ml feed/ml catalyst.h⁻¹ liquid hourly space velocity) of between about 5 and 25 h⁻¹ at a temperature of between about 350 and 550° C. Preferably, the feed stream may be contacted with the catalyst at a LHSV of between about 10 to 20 h⁻¹ at a temperature between about 420 and 500° C. The feed stream may include a C₅ to C₁₀ alpha olefin or mixtures thereof.

The feed stream may be contacted with the catalyst at a pressure of 100 Pa to 1 mPa, preferably between about 1 and 100 kPa, thus preferably between 0.1 atm to 10 atm.

Using the higher pH instead of the known low pH during the production of the catalyst has the following advantages. Firstly, it facilitates an uniform distribution of the active tungsten sites deposited on the carrier and secondly it lowers the Brφnsted acidity of the catalyst. The uniform distribution of the deposits improves the conversion rate, which in turn allows for a lower WO₃ loading, which in turn also lowers the Brφnsted acidity and the lower Brφnsted acidity in turn improves the selectivity of the catalyst towards linear olefin or primary metatheses products, particularly which of the metathesis of longer chained olefinic feed streams. These advantages are in addition to a WO₃/SiO₂ catalyst's inherent advantages.

According to a fourth aspect of the invention, there is provided a product produced by the process described above.

The product may include C₈ to C₂₀ internal olefins. The C₈ to C₂₀ internal olefins may be mostly linear.

The feed stream may predominantly be a linear alpha-olefin and the product may comprise of at least 4% of a corresponding primary metathesis product and at least 40% of a linear olefin product.

### DETAILED DESCRIPTION OF THE INVENTION

The invention is now described by way of example using 1-octene and 1-heptene as representative of olefinic feed streams.

It shall be understood that the examples are provided for illustrating the invention further and to assist a person skilled in the art with understanding the invention and are not meant to be construed as unduly limiting the reasonable scope of the invention.

Preparation of a Metathesis Catalyst:

Silica gel, Davisil grade 646 (surface area: 300 m²/g, pore volume: 1.15 cm³/g) was used as a carrier. A WO₃/SiO₂ precursor with a loading of 8 wt % WO₃ was prepared by wet impregnation of the silica carrier (13.8 g SiO₂) with an aqueous solution of ammonium metatungstatehydrate (Aldrich, 99.9%) of appropriate concentration (1.2752 g or 0.000431 moles of ammonium metatungstatehydrate). The pH of the aqueous solution of ammonium metatungstatehydrate (Aldrich, 99.9%) was adjusted to about 12 with the addition of either HNO₃ (Rochelle Chemicals, 55% chemically pure) or Ammoniumhydroxide (25% NH₄OH solution in water). The solution was stirred for 48 hours. This solution was then added to the carrier and the pH adjusted again. The mixture was stirred for 2 hours and the excess water was then removed by evaporation at 80° C. under reduced pressure. The residue was dried at 110° C. for 12 hours. The temperature was then raised at a rate of 1° C./min to 250° C. This temperature was maintained for 2 hours and then raised by 3° C./min to 550° C. The final step was a calcination step at 550° C. for 8 hours under an air atmosphere.

Catalysts with different tungsten metal loadings were prepared i.e. 3, 4.5, 6, 7, 8, 10, 15 and 20 wt % of WO₃ on SiO₃. These catalysts are characterised in Table 1.

TABLE 1

| | Actual | | | Avg. | |
|---|---|---|---|---|---|
| | Composition | Surface | Pore | Pore | |
| | (wt % | Area | Volume | Size | Crystallite |
| Catalyst | WO₃) | (m²/g) | (cm³/g) | (nm) | Size (Å) |
| 3% WO₃/SiO₂ | 2.8 | 326 | 1.11 | 13.7 | N/A |
| 4.5% WO₃/SiO₂ | 4.1 | 324 | 1.16 | 14.1 | N/A |
| 6% WO₃/SiO₂ | 5.5 | 312 | 1.12 | 14.1 | N/A |
| 7% WO₃/SiO₂ | 6.8 | 287 | 1.04 | 14.0 | N/A |
| 8% WO₃/SiO₂ | 8.0 | 252 | 0.98 | 15.6 | 126.0 |
| 10% WO₃/SiO₂ | 9.9 | 276 | 0.99 | 13.9 | 134.1 |
| 15% WO₃/SiO₂ | 14.2 | 267 | 0.96 | 14.0 | 141.3 |
| 20% WO₃/SiO₂ | 19.7 | 234 | 0.85 | 14.2 | 151.0 |

Optimisation of a Metathesis Catalyst Using 1-octene as a Representative Feed Stream:

Table 2 gives a summary of the conversions and product selectivities obtained with WO₃/SiO₂ metathesis catalysts with different WO₃ loadings using 1-octene as feed. All reactions were on-line for 8 hours and results are reported as averages over the 8-hour period. Reaction conditions were 460° C., 5.6 h⁻¹ LHSV and atmospheric pressure.

TABLE 2

| | WO₃ Loading/% | | | | | | | |
|---|---|---|---|---|---|---|---|---|
| | 3 | 4.5 | 6 | 7 | 8 | 10 | 15 | 20 |
| C₈ Conversion/ | 56.8 | 78.9 | 88.6 | 87.1 | 88.4 | 88.3 | 88.3 | 88.4 |
| % | | | | | | | | |
| Selectivity C₁₄ | 15.2 | 7.3 | 5.3 | 4.8 | 4.6 | 4.2 | 4.0 | 4.0 |
| Linear/% | | | | | | | | |
| Selectivity C₁₄ | 0.6 | 0.5 | 0.5 | 0.5 | 1.0 | 0.9 | 0.9 | 0.8 |
| Branched/% | | | | | | | | |
| Selectivity | 46.4 | 47.7 | 45.5 | 44.0 | 40.0 | 40.7 | 40.0 | 39.5 |
| C₉₋₁₃ | | | | | | | | |
| Linear/% | | | | | | | | |
| Selectivity | 1.6 | 2.2 | 2.6 | 2.7 | 5.7 | 5.8 | 5.4 | 5.3 |
| C₉₋₁₃ | | | | | | | | |
| Branched/% | | | | | | | | |

Graph 1 shows the relationship between WO₃ loading and C₈ conversion. It can be seen from Graph 1 that that WO₃ loading of more than about 6% wt % provides no significant increase in conversion.

Graph 2 shows the relationship between conversion and time (hours) for catalysts with different WO₃ loadings. It can be seen from Graph 2 that catalysts having a WO₃ loading of less than about 4.5% wt % experience significant poisoning.

These results indicate an optimum WO₃ loading where selectivity to linear metathesis products is high, branched product formation is relatively low and catalyst lifetime is high. This optimum appear to be in the region of between 6 and 8 wt % WO₃.

Graph 3 shows the effect of the variation of the pH during impregnation on catalyst selectivity towards primary metathesis products.

Tunnelling electron microscope analysis showed improved dispersion of WO₃ on the carrier with catalysts prepared with an aqueous solution at a high pH (pH10-12). More crystallites and an even or, in other words, uniform dispersion over the silica carrier was observed at a higher pH impregnation and Table 3 gives crystallite size determinations.

TABLE 3

| | pH of impregnation | Crystallite |
|---|---|---|
| Catalyst | solution | Size (A) |
| 8% WO₃/SiO₂ | 1 | 260.0 |
| 8% WO₃/SiO₂ | 5 | 126.0 |
| 8% WO₃/SiO₂ | 12 | 110.0 |

Table 4 gives a summary of the conversions and product selectivities obtained with WO₃/SiO₂ metathesis catalysts prepared by impregnating a silica carrier at different pH's, using 1-octene as feed. Impregnation at a higher pH resulted in increased production of linear metathesis products. The largest Improvement was observed with the production of the primary linear C₁₄ metathesis product. The linear secondary metathesis products also show an improvement with a higher pH. The reduction in branched metathesis products can be attributed to poisoning of some of the Brφnsted acidity necessary for skeletal isomerisation due to the basic environment during preparation.

TABLE 4

| | pH | | | | | |
|---|---|---|---|---|---|---|
| | 1 | 3 | 5 | 8 | 10 | 12 |
| Conversion/% | 88.5 | 88.7 | 88.2 | 88.2 | 87.6 | 85.5 |
| C₉-C₁₃ | 1.9 | 1.8 | 1.8 | 1.8 | 1.7 | 1.3 |
| Branched | | | | | | |
| C₉-C₁₃ Linear | 46.0 | 45.3 | 46.5 | 45.6 | 48.7 | 49.0 |
| C₁₄ branched | 0.3 | 0.3 | 0.3 | 0.3 | 0.3 | 0.2 |
| C₁₄ linear | 5.3 | 5.3 | 5.3 | 5.4 | 6.0 | 8.2 |

An example of Using the Optimised Catalyst in a Metathesis Process using a Feed Stream of 1-heptene:

Scheme 1 shows a plant equipped with the necessary work-up facilities and recycle lines for metathesis.

Column 1 includes a reboiler set at 220° C., Column 2 includes a condenser set at 25° C. and Reboiler set at 34° C. The Recycle line is set at 25° C., and the Reactor temperature is 460° C. The LHSV is 16 h⁻¹ (including a recycle loop of C₅-C₁₀ at a 1:5.6 ratio) and the Reactor pressure is 10 kPa_{g}, thus 0.1 atm. A C₇ single linear olefin stream was used as a feed stream to the reactor. The composition In mass % of the feed stream is depicted in Table 5.

TABLE 5

| | |
|---|---|
| 3-Me-3-hexene | 0.0984 |
| 5-Me-1-hexene | 0.0610 |
| 4-Me-1-hexene | 0.2029 |
| 2-Me-1-hexene | 1.0000 |
| 2-Methylhexane | 0.4711 |
| 3-Methylhexane | 1.5997 |
| 1-heptene | 74.6147 |
| n-heptane | 13.3506 |
| 2-methyl-2-hexene | 1.0000 |
| 3-Heptene | 1.1094 |
| diene or cyclic olefin | 1.4993 |
| 2-Heptene | 2.9874 |
| Dienes or cyclic olefins | ca 2.00 |

An 8 wt % WO₃/SiO₂ catalyst (0.3 mm average particle size, 20 ml) was loaded into a tubular fixed bed reactor (25.4 mm diameter). This catalyst was pre-treated at 550° C. under air (12 hours), followed by treatment at the same temperature with molecular nitrogen (12 hours) before allowing the catalyst to cool down under an inert atmosphere to the operating temperature (460° C.). The feed was introduced at 0.8 ml/min and the recycle line (containing the C₅-C₁₀ fraction) was operated at 4.5 ml/min. Samples of the purge stream, gas stream and heavy product (see Scheme 1) was analysed every 12 hours via a gas chromatograph. This was continued for 700 hours and the process terminated. The same catalyst was regenerated by a calcination step at 550° C. for 8 hours under an air atmosphere and a second run was started. The second run was continued for 1200 hours. In both cases, the catalyst was still active at the point of termination. A summary of the results obtained can be found in Table 6. Values presented are an average over 80% of the run duration, ignoring first and last 10% of the run. Ethylene purity is expressed as % ethylene in the methane—ethane—ethylene fraction. Propylene purity is expressed as % propylene in the propane—propylene fraction. The primary metathesis products of heptene are ethylene and dodecene, olefins formed outside this range can be referred to as secondary metathesis products. This value gives an indication of the ratio between isomerization of feed/product and metathesis on the catalyst surface.

TABLE 6

| | Run 1 | Run 2 |
|---|---|---|
| Online time (hours) | 700 | 1200 |
| Total feed conversion (%) | 88.2 | 84.7 |
| Selectivity (%) to | | |
| Ethylene | 7.56 | 8.52 |
| Propylene | 4.74 | 4.05 |
| Undecene (C₁₁) | 12.03 | 12.81 |
| Dodecene (C₁₂) | 41.28 | 46.25 |
| Tri-and Tetradecene (C₁₃-C₁₄) | 7.62 | 5.55 |
| Ethylene Purity (%) | 98.92 | 99.27 |
| Propylene Purity (%) | 98.85 | 98.97 |
| Linearity of C₁₂ (%) | 97.0 | 97.0 |
| % Primary metathesis | 65.3 | 66.6 |
| Mass Balance (%) | 89.4 | 95.1 |

The values of the two runs depicted in Table 6 are very similar. There was however a slight drop in conversion with the regenerated catalyst, but the selectivity towards the detergent range C₁₁ and C₁₂ increased with the regenerated catalyst.

The high linearity index of the dodecene is advantageous for example used in detergent synthesis. For linear alkyl benzenes synthesis the dodecene should be highly linear.

Optimising the Above Example Process using as an Example, a Feed Stream of 1-heptene:

By using the abovementioned optimised catalyst together with the optimised conditions the applicant managed to keep the catalyst on-line for 1200 hours without losing any significant activity or any significant indication of catalyst deactivation. The catalyst is therefore capable of running for longer than 1200 hours. Prior art work done on short-chain olefins (C₂-C₄) only managed a maximum of 40% conversion that could be kept constant for 60 hours before deactivation started to occur. (E. D. Oliver, Butylenes, Process Economics Program SRI Report, October 1971, Report No 71)

Optimisation of reaction conditions with respect to pressure, temperature and contact time of the feed stream with catalyst (LHSV) is equally as important as developing and optimising the right catalyst for a specific chemical transformation.

Optimising Temperature and LHSV

Graph 4 shows that, with an 8 wt % WO₃/SiO₂ catalyst, by increasing the temperature and decreasing the LHSV at 10 kPa_{g}, the conversion can be increased. However, due to side reactions, the conversion observed is not necessarily conversion towards metathesis products. Temperature and LHSV alone should therefore not be used to find the optimised reaction conditions.

The applicant also realised that selectivity towards the C₁₁-C₁₄ range can also result in the wrong optimised reaction conditions, as selectivity does not take the conversion into account. A high selectivity can be obtained with a low conversion, which means that a fairly high recycle stream to feed stream ratio must be employed which may not make economical sense. The applicant found that a high LHSV and low temperature must be employed in order to give the highest selectivity towards a C₁₁-C₁₄ range, see Graph 5. Taking into account the low conversion under these conditions, the applicant concluded that selectivity towards a C₁₁-C₁₄ range should not be used as a probe for condition optimisation.

However, the yield towards C₁₁-C₁₄ on the other hand does incorporate both conversion and selectivity as can be seen from equation 1.

$$Yield = \frac{Conversion \times Selectivity}{100} \qquad ( 1 )$$

By using yield towards the C₁₁-C₁₄ range, the applicant was able to arrive at a solution for the reaction condition optimisation. This was done through a three-stage design as depicted in Graph 6, giving the direction of increase over each design block. The result of a combination of all three design blocks can be seen in Graph 7, resulting in an optimum yield at 460° C. and a total LHSV of 16 h⁻¹.

Optimising Temperature, LHSV and Pressure.

The applicant realised that an increase in pressure causes and increase in contact time of the feed with the catalyst and effectively decreasing the LHSV and as thus it may be appreciated that an increase in pressure on the system would lower the yield. This was indeed found to be the case as depicted in Graph 8 (units in Graph 8 indicated as atmospheric pressure). Although a pressure below atmospheric pressure will provide a better yield, economical and practical considerations prompted the applicant to choose atmospheric pressure as an optimum.

The applicant therefore found that the optimum yield towards the C₁₁-C₁₄ range can be obtained by working at a temperature of 460° C., a LHSV of 16 h⁻¹ and 10 kPa_{g} pressure, thus 1 atm.

Table 7 gives a comparison of experimental results of a 6 wt % WOSiO₂ catalyst with the 8 wt % WO₃/SiO₂ catalyst over a 48 hour period at the abovementioned optimised conditions.

TABLE 7

| Catalyst | 8% WO₃/SiO₂ | 8% WO₃/SiO₂ |
|---|---|---|
| LHSV | 16 | 16 |
| Conversion/% | 54.2 | 71.3 |
| Selectivity C₉-C₁₃ Branched/% | 1.9 | 1.8 |
| Selectivity C₉-C₁₃ Linear/% | 47.6 | 47.3 |
| Selectivity C₁₄ Branched/% | 0.2 | 0.2 |
| Selectivity C₁₄ Linear/% | 14.7 | 15.2 |
| % Branched C₉-C₁₃ | 4.1 | 3.9 |
| % Branched C₁₄ | 1.4 | 1.4 |

1-25. (canceled)

26. A method of preparing a metathesis catalyst, the method including the steps of:
mixing an aqueous solution of transition metal anions having a pH of 9 or higher with a carrier; and
removing water from the mixture by means of evaporation.

27. A method as claimed in claim 26, wherein the carrier is silica and the transition metal is tungsten.

28. A method as claimed in claim 27, wherein the aqueous solution contains tungsten in the form of ammonium metatungstatehydrate and/or ammonium tungstate.

29. A method as claimed in claim 38, wherein the aqueous solution contains tungsten in the form of ammonium metatungstatehydrate and wherein the concentration of the ammonium metatungstatehydrate and the mass of the silica are selected such that the WO₃ on the SiO₂ is from 4 to 10 wt %.

30. A method of preparing a metathesis catalyst as claimed in claim 26, wherein excess water is removed by evaporation at about 80° C. under reduced pressure to form a residue.

31. A method of preparing a metathesis catalyst as claimed in claim 30, wherein further water is removed after removal of the excess water by drying the residue at about 110° C. for about 12 hours, then by raising the temperature at a rate of about 1° C. every minute up to about 250° C., maintained at about 250° C. for about two hours and then by raising the temperature at a rate of about 3° C. every minute up to about 550° C.

32. A method of preparing a metathesis catalyst as claimed in claim 31, wherein the residue is then calcined.

33. A method of preparing a metathesis catalyst as claimed in claim 32, wherein the residue is calcined at about 550° C. for about 8 hours.

34. A method of preparing a metathesis catalyst as claimed in claim 32, wherein the residue is calcined at a temperature and for duration such that the calcination step substantially removes NH₃, ensures that the oxidation state of the tungsten is mostly 6+ and ensures that the tungsten oxide is bound to the carrier.

35. A catalyst for metathesis of an olefinic feed stream, which includes:
a transistion metal oxide; and
a carrier, the transition metal oxide being deposited onto the carrier from an aqueous solution of transition metal oxide anions at a pH of 9 or more.

36. A catalyst as claimed in claim 35, wherein the transition metal is tungsten and the carrier is silica.

37. A catalyst as claimed in claim 35, wherein the catalyst is a heterogeneous catalyst.

38. A catalyst as claimed in claim 36, wherein the catalyst is a heterogeneous catalyst.

39. A catalyst as claimed in claim 37, wherein most of the tungsten oxide deposits are substantially amorphous.

40. A catalyst as claimed in claim 38, wherein most of the tungsten oxide deposits are substantially amorphous.

41. A catalyst as claimed in claim 36, wherein the catalyst is characterised in that at least a portion of some of the tungsten oxide deposits are in the form crystallites of less than about 135 Å across on the surface of the carrier.

42. A catalyst as claimed in claim 36, wherein the tungsten oxide is from about 4 to 10 wt % on SiO₂.

43. A catalyst as claimed in claim 36, wherein the catalyst is characterised in that it remains catalytically active for at least 1000 hours at optimal operating conditions.

44. A catalyst as claimed in claim 36, wherein the catalyst is characterised in that it provides a conversion rate of at least 30% for at least 50 hours at optimal operating conditions.

45. A metathesis process, which includes the step of:
contacting a C₅ and/or higher olefinic feed stream with a catalyst for metathesis at a temperature of between 350° C. and 600° C., which catalyst includes:
a transistion metal oxide; and
a carrier, the transition metal oxide being deposited onto the carrier from an aqueous solution of transition metal oxide anions at a pH of 9 or more.

46. A metathesis process as claimed in claim 45, wherein the transition metal is tungsten and the carrier is silica.

47. A metathesis process as claimed in claim 45, wherein the process includes a step of activating the catalyst about 500 to 700° C. for about 8 hours in an inert atmosphere.

48. A metathesis as claimed in claim 47, wherein the olefinic feed stream is selected such that the process yields C₁₀ to C₁₈ olefins.

49. A metathesis process as claimed in claim 45, wherein the feed stream is contacted with the catalyst at a LHSV of between 5 and 25 h⁻¹ at a temperature of between 350 and 550° C.

50. A metathesis process as claimed in claim 45, wherein the feed stream is contacted with the catalyst at a pressure of 0.1 to 10 atm.
